(19) United States
(12) Patent Application Publication
(10) Pub. No.: US 2005/0159431 A1
(43) Pub. Date: Jul. 21, 2005

(54) Substituted alkyl uracils and thereof

(75) Inventors: Barbara Albrecht, Leverkusen (DE); Michael Gerisch, Wuppertal (DE); Michael Harter, Leverkusen (DE); Thomas Krahn, Hagen (DE); Felix Oehme, Wuppertal (DE); Karl-Heinz Schlemmer, Wuppertal (DE); Henning Steinhagen, Sulzbach (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/501,033

(22) PCT Filed: Jan. 3, 2003
(86) PCT No.: PCT/EP03/00027

(30) Foreign Application Priority Data
Jan. 15, 2002 (DE) 102 01 240.7

(51) Int. Cl. A61K 31/519; A61K 31/517; C07D 491/02; C07D 498/02

(52) U.S. Cl. 514/260.1; 514/266.3; 544/278; 544/285

(57) ABSTRACT

The invention relates to novel compounds of formula (I), to a method for the production thereof, and to their use as medicament active ingredients for the prophylaxis and/or treatment of diseases.

The present invention relates to novel chemical compounds, to a process for their preparation and to their use as medicaments, in particular for the prophylaxis and/or treatment of ischemia and reperfusion damage.

The elucidation of the molecular mechanism of cell death is the subject of intense biomedical research efforts. The aim is to find specifically active compounds which have modulating action in this process. When the individual biochemical steps resulting in cell death were examined, attention was drawn to poly(ADP-ribose)-synthetase (PARS), a protein which is expressed strongly in the cell nucleus and which is involved in deoxyribonucleic acid (DNA) damage repair [Szabo and Dawson, Trends in Pharmacological Sciences, 19, 287-298 (1998)].

Activation of PARS plays an important role in N-methyl-D-aspartate (NMDA)- and NO-induced neurotoxicity [Zhang et al., Science, 263, 687-689 (1994); Wallis et al., NeuroReport, 5, 245-248 (1993)], cerebral ischemia [Endres et al., J. Cereb. Blood Flow Metabol., 17, 1143-1151 (1997)], traumatic brain injuries [Wallis et al., Brain Res., 710, 169-177 (1996)] and ischemia/reperfusion damage to heart and skeletal muscle [Thiemermann et al., Proc. Nat. Acad. Sci., 94, 679-683 (1997)]. In addition, inhibition of PARS appears to have a positive effect on the therapy of arthritis [Szabo et al., Japanese J. Pharm., 75, Supp. I:102 (1997)], diabetes [Shimabukuro et al., J. Clin. Invest., 100, 290-295 (1997)] and endotoxic or septic shock [Zingarelli et al., Shock, 5, 258-264 (1996)], radiosensitization of hypoxic tumor cells [Weltin et al., Oncol. Res., 6, 399-403 (1994)], chronic colitis [Jijon et al., Am. J. Physiol. Gastrointest. Liver Physiol., 279, G641-51 (2000)], sudden deafness [Tabuchi et al., Ann. Otol. Rhinol. Laryngol., 110(2), 118-21 (2001)], inflammatory pulmonary disorders, such as, for example, asthma and chronic bronchitis [Cuzzocrea et al., Eur. J. Pharm., 342, 67-76 (1998)] and cancer.

PARS, an enzyme which constructs polymeric ADP-ribose units from nicotinamide adenosine dinucleotide (NAD⁺) as substrate, is activated when the DNA is damaged by single- or double-strand breaks. The polymeric ADP-ribose units formed are attached both to PARS itself and to other proteins, for example histones, topoisomerases and polymerases.

Increased activation of PARS results in a massive NAD⁺ consumption. The strong decrease of the NAD⁺ concentration and the resulting impediment of ATP synthesis (decrease of the ATP concentration) causes deterioration of the energetic state of the cell, which may lead to premature cell death (necrosis).

In the heart, reperfusion of ischemic myocardium results in the generation of radicals, neutrophil infiltration, destruction of the myocardial tissue structure, contraction dysfunctions and necrosis. The H₂O₂ generated during the reperfusion phase reacts rapidly with NO, forming peroxynitrite. NO, peroxynitrite and H₂O₂ cause DNA strand breaks, thus resulting in overstimulation of PARS.

A further important point in the case of reperfusion damage is the accumulation of neutrophils in the reperfused myocardium. Activation of PARS increases the infiltration of neutrophils by stimulating the expression of P-selectin and ICAM-1.

Healthy PARS knock-out mice capable of reproduction are substantially protected against reperfusion damage. Infiltration of neutrophils is reduced by 50% and the structure of the myocardial tissue remains intact during the reperfusion phase.

In cases of ischemia and reperfusion damage to the heart and brain, low-molecular-weight PARS inhibitors, such as, for example, 3-aminobenzamide and 1,5-dihydroxyisoquinoline, protect the tissue against necrotic cell death (reduction of the infarct size by 30 to 48%) and delay myocardial and neuronal dysfunction.

However, the PARS inhibitors hitherto tested in animal experiments have various disadvantages. Thus, for example, 3-aminobenzamide is an unspecific PARS inhibitor which also inhibits cytochrome P₄₅₀ (Eriksson et al., Toxicology and applied Pharmacology, 136, 324-331 (1996)); in contrast, 5-iodo-6-amino-1,2-benzopyrone has serious side-effects (Szabo and Dawson, Trends in Pharmacol. Sciences, 19, 287-298 (1998)). Moreover, most inhibitors are not very potent and are therefore only efficacious in animals at a relatively high dosage (Thiemermann et al., Proc. Natl. Acad. Sci., 94, 679-683.(1997)).

JP-A-032645679 and Chem. Pharm. Bull. 38 (10), 2726-2732 (1990) disclose bicyclic 2,4-(1H,3H)-pyrimidinediones as 5-HT₂ antagonists for the treatment of cardiovascular diseases, depression and other mental disorders. U.S. Pat. No. 5,859,014 discloses tetrahydroquinazolinedione derivatives as α₁ adrenergic receptor antagonists for the treatment of prostate hypertrophy. WO-A-00/42025 describes dihydropyrimidinones as PARS inhibitors. DE-A-1959705 and DE-A-2126148 list uracil derivatives for preparing crop protection agents. DE-A-2142317 mentions uracil derivatives having hypnotic properties. Furthermore, various bridged uracils are described in the literature as nucleoside analogues with potential antiviral action (for example Nucleosides Nucleotides 13 (1-3), 177-196; 13 (4), 891-902 (1994) and J. Med. Chem. 39 (3), 789-795 (1996)).

Accordingly, it is an object of the present invention to provide novel substances for the prophylaxis and/or treatment of disorders, in particular of ischemia and reperfusion damage.

Here, the compounds according to the invention act as inhibitors of poly(ADP-ribose)-synthetase (PARS).

The present invention relates to compounds of formula (I) 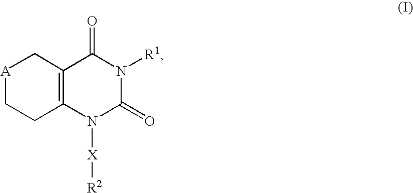
in which
A represents —CH₂—, —O— or —S—,
R¹ represents hydrogen or alkoxycarbonyl,
R² represents aryl or heteroaryl which for their part may be substituted up to three times, independently of one another, by substituents selected from the group consisting of nitro, halogen, cyano, aryl, hetaryl, benzyl, alkyl, cycloalkyl, alkoxy, formyl, alkoxycarbonyl, trifluoromethyl, di- and trifluoromethoxy, hydroxyl, amino, alkylamino, aminosulfonyl, alkylsulfonylamino, arylsulfonylamino, hetarylsulfonylamino, —Y—OR³ and —Y—NR³R⁴,
   in which
   Y represents CH₂, C(═O) or —NH—C(═O)—CHR⁵—,
      in which * represents the point of attachment to the aromatic or heteroaromatic radical,
   R³ and R⁴ independently of one another represent hydrogen, optionally hydroxyl- or amino-substituted alkyl, alkenyl or alkoxycarbonyl or
   R³ and R⁴ together with the nitrogen atom to which they are attached form a 5- to 7-membered heterocycle which may contain a further heteroatom N, O or S in the ring and which is optionally substituted by amino, hydroxyl, alkoxycarbonyl or alkyl which for its part may be substituted by hydroxyl or amino,
   R⁵ represents hydrogen or alkyl which for its part may be substituted by phenyl, 4-hydroxyphenyl, amino, hydroxyl, carboxyl, guanidino, imidazolyl, indolyl, mercapto or methylthio, or
   R³ and R⁵ together represent propane-1,3-diyl or butane-1,4-diyl, and
X represents alkanediyl in which one methylene group may be replaced by an oxygen atom.

The compounds according to the invention can also be present in the form of their salts, solvates or solvates of the salts.

Depending on their structure, the compounds according to the invention can exist in stereoisomeric forms (enantiomers, diastereomers). Accordingly, the invention also relates to the enantiomers or diastereomers and to their respective mixtures. From such mixtures of enantiomers and/or diastereomers, the stereoisomerically uniform components can be isolated in the known manner.

Depending on the structure of the compounds, the invention also relates to tautomers of the compounds.

Preferred salts in the context of the invention are physiologically acceptable salts of the compounds according to the invention.

Physiologically acceptable salts of the compounds (I) include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene-disulfonic acid, acetic acid, propionic acid, lactic acid, tartaric acid, malic acid, citric acid, fumaric acid, maleic acid and benzoic acid.

Physiologically acceptable salts of the compounds (I) also include salts of customary bases, such as, by way of example and by way of preference, alkali metal salts (for example sodium and potassium salts), alkaline earth metal salts (for example calcium and magnesium salts) and ammonium salts, derived, from ammonia or organic amines having 1 to 16 carbon atoms, such as, by way of example and by way of preference, ethylamine, diethyl amine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabiethylamine, arginine, lysine, ethylenediamine and methylpiperidine.

Solvates in the context of the invention are those forms of the compounds which, in the solid or liquid state, form a complex by coordination with solvent molecules. Hydrates are a specific form of the solvate where the coordination is with water.

In, the context of the present invention, the substituents are, unless specified otherwise, as defined below:

Alkyl per se and “alk” and “alkyl” in alkoxy, alkylamino alkylsulfonylamino and alkoxycarbonyl denote a linear or branched alkyl radical having generally 1 to 6, preferably 1 to 4, particularly preferably 1 to 3, carbon atoms, by way of example and by way of preference methyl, ethyl, n-propyl, isopropyl, tert-butyl, n-pentyl and n-hexyl.

Alkoxy denotes, by way of example and by way of preference, methoxy, ethoxy, n-propoxy, isopropoxy, tert-butoxy, n-pentoxy and n-hexoxy.

Alkylamino denotes an amino radical having one or two alkyl substituents chosen independently of one another, by way of example and by way of preference methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylsulfonylamino denotes, by way of example and by way of preference, methylsulfonylamino, ethylsulfonylamino, n-propylsulfonylamino, isopropyl-sulfonylamino, tert-butylsulfonylamino, n-pentylsulfonylamino and n-hexyl-sulfonylamino.

Alkoxycarbonyl denotes, by way of example and by way of preference, methoxycarbonyl, ethoxycarbonyl, n-propoxycarbonyl, isopropoxycarbonyl, tert-butoxycarbonyl, n-pentoxycarbonyl and n-hexoxycarbonyl.

Alkanediyl denotes a straight-chain or branched alkanediyl radical having generally 1 to 6, preferably 1 to 4, carbon atoms, by way of example and by way of preference. methylene, ethane-1,2-diyl, propane-1,2-diyl, propane-1,3-diyl, propane-2,2-diyl, butane-1,3-diyl, butane-1,4-diyl, butane-2,4-diyl, pentane-2,4-diyl, 2-methylpentane-2,4-diyl.

If a methylene group of the alkanediyl radical is substituted by an oxygen atom, the following radicals may be mentioned by way of example and by way of preference: 3-oxabutane-1,4-diyl, 4-oxabutane-1,4-diyl, 3-oxapentane-1,5-diyl, 4-oxapentane-1,5-diyl, 4-oxahexane-1,6-diyl.

Alkenyl denotes a straight-chain or branched alkenyl radical having generally 2 to 6, preferably 2 to 4, particularly preferably 2 or 3, carbon atoms, by way of example and by way of preference vinyl, allyl, n-prop-1-en-1-yl, n-but-2-en-1-yl.

Cycloalkyl denotes a cycloalkyl group having generally 3 to 8, preferably 5 to 7, carbon atoms, by way of example and by way of preference cyclopropyl, cyclobutyl, cyclopentyl, cyclohexyl and cycloheptyl.

Aryl per se and “aryl” in arylsulfonylamino denotes a mono-, bi- or tricyclic aromatic carbocyclic radical having generally 6 to 14 carbon atoms; by way of example and by way of preference phenyl, naphthyl and phenanthrenyl.

Arylsulfonylamino denotes, by way of example and by way of preference, phenylsulfonylamino, naphthylsulfonylamino and phenanthrenylsulfonylamino.

Heteroaryl per se and “hetaryl” in hetarylsulfonylamino denotes an aromatic, optionally benzo-fused radical having generally 5 or 6 ring atoms and up to 3 heteroatoms from the group consisting of S, O and N, by way of example and by way of preference thienyl, furyl, pyrrolyl, thiazolyl, oxazolyl, pyrazolyl, imidazolyl, isoxazolyl, isothiazolyl, pyridyl, pyrimidyl pyridazinyl, pyrazinyl, indolyl, indazolyl, benzofuranyl, benzothiophenyl, quinolinyl, isoquinolinyl.

Hetarylsulfonylamino denotes, by way of example and by way of preference, pyridylsulfonylamino, thienylsulfonylamino and pyrazolylsulfonylamino.

Halogen denotes fluorine, chlorine, bromine and iodine.

Preference is given to compounds of the formula (I),
in which
A represents —CH₂— or —S—,
R¹ represents hydrogen,
R² represents phenyl, pyridyl, pyrazolyl or imidazolyl which for their part may be substituted up to three times, independently of one another, by substituents selected from the group consisting of nitro, halogen, phenyl, benzyl, (C₁-C₄)-alkyl, (C₁-C₄)-alkoxy, formyl, (C₁-C₄)-alkoxycarbonyl, amino, hydroxyl, aminosulfonyl and —Y—NR³R⁴,
   in which
   Y represents CH₂, *—NH—C(═O)—CH₂— or *—NH—C(═O)—CH(CH₃)—,
      in which * represents the point of attachment to the aromatic or heteroaromatic radical,
   R³ and R⁴ independently of one another represent hydrogen, optionally hydroxyl- or amino-substituted (C₁-C₄)-alkyl, (C₂-C₄)-alkenyl or (C₁-C₄)-alkoxycarbonyl or
   R³ and R⁴ together with the nitrogen atom to which they are attached form a 5- to 7-membered heterocycle which may contain a further heteroatom N or O in the ring and which is optionally substituted by amino, hydroxyl, (C₁-C₄)-alkoxycarbonyl or (C₁-C₄)-alkyl which for its part may be substituted by hydroxyl or amino, and
X represents (C₁-C₄)-alkanediyl.

Particular preference is given to compounds of the formula (I),
in which
A represents —S—,
R¹ represents hydrogen,
R² represents phenyl or imidazolyl which for their part may be substituted up to three times, independently of one another, by substituents selected from the group consisting of nitro, fluorine, chlorine, bromine, methyl, ethyl, isopropyl, methoxycarbonyl and —Y—NR³R⁴,
   in which
   Y represents CH₂ or *—NH—C(═O)—CH₂—,
      in which * represents the point of attachment to phenyl or imidazolyl,
   R³ and R⁴ independently of one another represent hydrogen, methyl, ethyl, isopropyl which are optionally substituted by hydroxyl or amino, or represent allyl or methoxycarbonyl,
   or
   R³ and R⁴ together with the nitrogen atom to which they are attached represent pyrrolidin-1-yl, piperidin-1-yl, piperazin-1-yl, 4-methyl-piperazin-1-yl, 4-(2-hydroxyethyl)piperazin-1-yl or morpholin-4-yl and
X represents ethane-1,2-diyl; propane1,3-diyl or butane-1,4-diyl.

The present invention also provides a process for preparing the compounds of the formula (I) where
compounds of the formula (II) 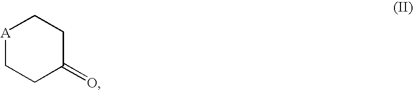
   in which
A is as defined above,
are reacted with compounds of the formula (III)
   H₂N—X—R² (III),
   in which
X and R² are as defined above,
to give compounds of the formula (IV) 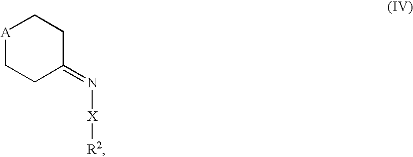
   in which
A, X and R² are as defined above,
then reacted with chlorocarbonyl isocyanate to give compounds of the formula (Ia) 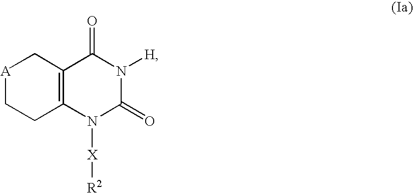
   in which
A, X and R² are as defined above and R¹ represents hydrogen,
and compounds of the formula (Ia) are, if appropriate, reacted with compounds of the formula (V)
   R¹-Z (V),
   in which
R¹ is as defined above, but is not hydrogen, and Z represents a leaving group,
to give compounds of the formula (I) in which R¹ is not hydrogen.

The resulting compounds of the formula (I) can then be subjected to further derivatizations carried out by customary methods.

The compounds of the formula (I) obtained in this manner can then, if appropriate, be converted into the corresponding salts, for example by reaction with an acid.

The process according to the invention for preparing compounds of the formula (I) can be illustrated in an exemplary manner by the formula scheme below:

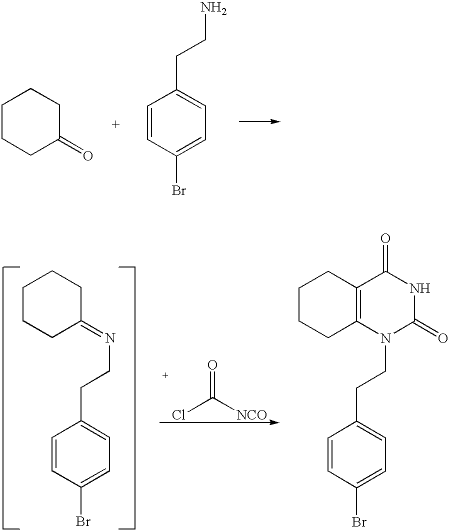

The compounds of the formula (III) are commercially available, known from the literature or can be prepared by customary methods or analogously to the reaction steps described in the examples. If R² is heteroaryl which is attached via a nitrogen atom, compounds of the formula (III) can be prepared, for example,
by reacting compounds of the formula (VI)
   R²—H (VI),
   in which
R² represents heteroaryl which is attached via a nitrogen atom to the hydrogen atom,
with compounds of the formula (VII) 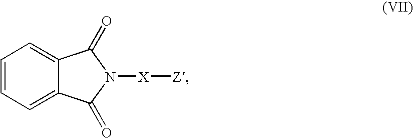
   in which
X is as defined above and Z¹ represents a leaving group,
to give compounds of the formula (VIII) 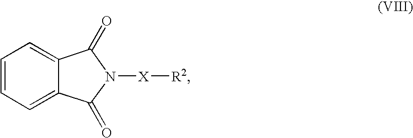
   followed by removal of the phthalimide group.

The reaction sequence is illustrated by the reaction scheme below:

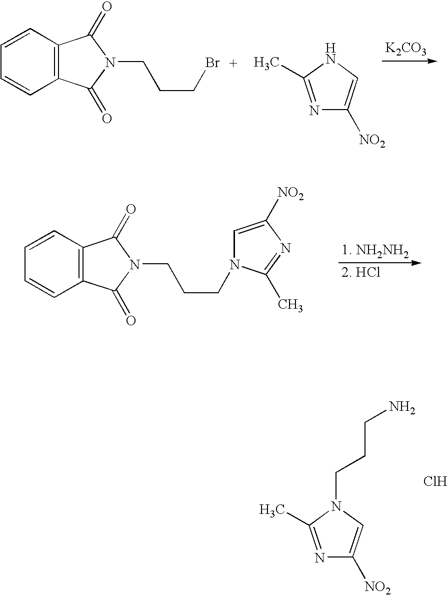

Suitable solvents for the processes described above are organic solvents which are inert under the reaction conditions, or water. These include halogenated hydrocarbons, such as dichloromethane, trichloromethane, carbon tetrachloride, 1,2-dichloroethane, trichloroethane, tetrachloroethane, 1,2-dichloroethylene or trichloroethylene, ethers, such as diethyl ether, dioxane, tetrahydrofuran, glycol dimethyl ether or diethylene glycol dimethyl ether, hydrocarbons, such as benzene, xylene, toluene, hexane or cyclohexane, or other solvents, such as dimethyl-formamide, dimethyl sulfoxide, N-methylpyrrolidone, acetonitrile or pyridine, or mixtures thereof.

The reactions are generally carried out in a temperature range of from −78° C. to 150° C.

The reactions can be carried out under atmospheric pressure, elevated pressure or reduced pressure (for example in the range from 0.5 to 5 bar). In general, the reactions are carried out under atmospheric pressure.

Suitable bases are the customary inorganic or organic bases. These preferably include alkali metal and alkaline earth metal hydroxides, such as, for example, lithium, sodium hydroxide or potassium hydroxide, or alkali metal and alkaline earth metal carbonates, such as sodium carbonate or potassium carbonate, or sodium methoxide or potassium methoxide or sodium ethoxide or potassium methoxide or potassium tert-butoxide, or amides, such as sodium amide, lithium bis(trimethylsilyl)amide or lithium diisopropylamide, or amines, such as triethylamine, diisopropylethylamine, diisopropylamine, N-methylmorpholine, 4-dimethylaminopyridine or pyridine.

The reaction step (II)+(III)→(IV) is preferably carried out in the solvent toluene. For this reaction, the temperature range is in particular between 80° C. and 120° C. Moreover, the reaction can, if required, be accelerated by addition of catalytic amounts of acid, preferably an organic sulfonic acid, in particular camphorsulfonic acid.

The reaction of compounds (IV) with chlorocarbonyl isocyanate to give compounds (Ia) is preferably carried out in the solvent toluene. Here, the addition of chlorocarbonyl isocyanate is preferably carried out at room temperature, further reaction is then carried out in particular in a temperature range between 80° C. and 120° C.

In the reaction (Ia)+(V)→(I), a suitable leaving group Z for compounds of the formula (V) is, for example: halogen or 1-imidazolyl. Preference is given to chlorine.

The reaction (VI)+(VII)→(VIII) is preferably carried out in the solvent dimethyl-formamide using the base potassium carbonate. The preferred temperature range for this reaction is between 20° C. and 130° C. Suitable leaving groups Z′ in compounds of the formula (VII) are, for example, halogen, mesylate, tosylate or triflate; preference is given to bromine.

The compounds of the formula (VIII) obtained in the reaction above are, preferably in the solvent ethanol and using aqueous hydrazine hydrate solution in a temperature range of from 50° C. to 80° C., reacted further, with removal of the phthalimide group. By adding an acid, preferably a hydrochloric acid, the amines of the formula (III) can be obtained in the form of their salts.

The compounds of the formulae (II), (V), (VI) and (VII) are commercially available, known from the literature or can be prepared by customary methods or analogously to the reaction steps described in the examples.

Surprisingly, the compounds of the formula (I) have an unforeseeable useful spectrum of pharmacological and pharmacokinetic activity, and they are therefore particularly suitable for the prophylaxis and/or treatment of disorders in humans and animals.

Owing to their pharmacological properties, the compounds according to the invention can be used on their own or in combination with other active compounds, preferably for the prophylaxis and/or treatment of ischemic and reperfusion damage in the heart (after an acute infarction), in the brain (after a stroke) or in skeletal muscle, for cardiovascular disorders, such as, for example, unstable angina pectoris and arteriosclerosis, neuronal and neurodegenerative disorders, such as, for example, epilepsy, chronic pain, Alzheimer's disease and Parkinson's disease, traumatic brain injuries, septic shock, and also arthritis, diabetes, chronic colitis, sudden deafness, inflammatory pulmonary disorders, such as, for example, asthma and chronic bronchitis, and cancer.

The present invention also relates to medicaments comprising at least one compound according to the invention, preferably together with one or more pharmaceutically acceptable auxiliaries, and to their use for the purposes mentioned above.

The present invention furthermore relates to a method for the prophylaxis and/or treatment of the clinical pictures mentioned above using the substances of the formula (I).

In addition, the compounds according to the invention can be used for the treatment of acute myocardial infarction, including in combination with one or more of the following medicaments which are used for the standard therapy of acute myocardial infarction: calcium canal blockers (such as, for example, nifedipine, diltiazem, verapamil), nitrovasodilators (such as, for example, isosorbide dinitrate, glycerol trinitrate, isosorbide 5-mononitrate, molsidomine), beta blockers (such as, for example, metoprolol, atenolol, propranolol, solatol), platelet aggregation inhibitors (such as, for example, acetylsalicylic acid, triclopidine, clopidrogrel), thrombolytics (fibrinolytics) (such as, for example, streptokinase, alteplase, reteplase, urokinase, anistreplase), anticoagulants (such as, for example, heparin, warfarin, phenprocoumarin, low-molecular-weight heparins), ACE inhibitors (such as, for example, enalapril), glycoprotein IIb/IIIa receptor antagonists (such as, for example, tirofiban, eptifibatid), antiarrhythmics (such as, for example, lidocaine, amiodarone) and beta-adrenergic agonists (such as, for example, dopamine, dobutamine).

The active compound can act systemically and/or locally. To this end, it can be administered in a suitable manner, such as, for example, orally, parenterally, pulmonarily, nasally, sublingually, lingually, buccally, rectally, transdermally, conjunctivally, otically or as an implant, for example in the form of an active compound-containing stent.

For these administration routes, the active compound can be administered in suitable administration forms.

Administration forms suitable for oral administration are known administration forms which release the active compound rapidly and/or in modified form, such as, for example, tablets (uncoated and also coated tablets, for example enterically coated tablets or film-coated tablets), capsules, sugar-coated tablets, granules, pellets, powders, emulsions, suspensions, solutions and aerosols.

Parenteral administration can be effected by circumventing a bioabsorption step (in an intravenous, intraarterial, intracardial, intraspinal or intralumbal manner), or via bioabsorption (intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates and sterile powders.

Medicinal forms suitable for the other administration routes are, for example, medicinal forms for inhalation (inter alia powder inhalators, nebulizers), nasal drops/solutions, sprays; capsules or tablets to be administered lingually, sublingually or buccally, suppositories, preparations for ears or eyes, vaginal capsules, aqueous suspensions (lotions, agitated mixtures), lipophilic suspensions, ointments, creams, milk, pastes, powder for spreading or implants.

The active compounds can be converted in a manner known per se into the administration forms listed. This is effected using pharmaceutically suitable auxiliaries. These include, inter alia, excipients (for example microcrystalline cellulose), solvents (for example liquid polyethylene glycols), emulsifiers (for example sodium dodecyl sulfate), dispersants (for example polyvinylpyrrolidone), synthetic and natural biopolymers (for example albumin), stabilizers (for example antioxidants such as ascorbic acid), colorants (for example inorganic pigments, such as iron oxides), or flavor- and/or odor-masking substances.

In the pharmaceutical preparations listed above, the therapeutically active compounds should be present in a concentration of from about 0.1 to 99.5, preferably from about 0.5 to 95, % by weight of the total mixture, i.e. the active compound should be present in amounts sufficient to achieve the dosage range indicated.

In general, it has been found to be advantageous both in human and veterinary medicine to administer the active compound(s) according to the invention in total amounts of from about 0.01 to about 100, preferably from 0.05 to 50, mg/kg of body weight per 24 hours, if appropriate in the form of a plurality of individual doses, to obtain the desired results. An individual dose preferably comprises the active compound(s) according to the invention in amounts of from about 0.01 to 50, in particular from 0.1 to 10, mg/kg of body weight.

In spite of this, it may be necessary, if appropriate, to depart from the amounts mentioned, namely depending on the body weight or the administration route, on the individual response to the medicament, the manner of its formulation and the time or interval at which administration takes place. Thus, in some cases it may be adequate to manage with less than the abovementioned minimum amount, while in other cases the upper limit mentioned has to be exceeded. If relatively large amounts are administered, it may be advisable to divide these into a number of individual administrations over the day.

Unless indicated otherwise, all percentages in the tests and examples below are based on weight; parts are parts by weight. Solvent ratios, dilution ratios and stated concentrations of liquid/liquid solutions are in each case based on volume.

A Evaluation of the Physiological Activity

1) Test Description PARS Inhibition Test (In Vitro)

The activity of substances as PARS inhibitors is tested in accordance with the method of Ushiro [Ushiro et al., J. Biol. Chem., 262, 2352-2357 (1987)]. To this end, recombinantly expressed (Bac-To-Bac, Baculo virus expression system; Instruction Manual; Life Technologies) human PARS enzyme is activated in a buffer which contains radioactively labeled [¹⁴C]-NAD⁺. The poly(ADP-ribose) units that are synthesized are precipitated using trichloroacetic acid, and the proportion of labeled protein is determined by scintillation measurements. Incubation of PARS with inhibitors leads to a decrease in the proportion of labeled protein and thus to a reduced radioactivity.

Inhibition of the PARS activity can be represented as a percentage of PARS inhibition in incubation with different substances or as the concentration at which 50% of the enzyme is inhibited, i.e. as IC₅₀ value.

Material

| | | |
|---|---|---|
| Buffer: | 100 mM | 2-amino-2-hydroxymethyl-1,3-propanediol (tris)-HCl, |
| | | pH 7.4 |
| | 10 mM | MgCl₂ |
| | 1 mM | dithiothreitol (DTT) |

| | |
|---|---|
| | Tris-HCl and MgCl₂ are dissolved in water, DTT is added from an |
| | aqueous 100 mM stock solution (stored at −20° C.) and the pH is |
| | adjusted with concentrated HCl to 7.4. |
| DNA: | 1 mg/ml of calf thymus DNA |
| | 1 mg/ml of calf thymus DNA (from Sigma) is dissolved in water and |
| | sonicated to induce strand breaks. 500 μl aliquots were stored at |
| | −20° C. |
| Histones: | 10 mg/ml of type IIA histones, calf thymus |
| | 10 mg/ml of lyophilized histones (from Sigma) are dissolved in water. |
| | 500 μl aliquots are stored at −20° C. |
| NAD⁺ Mix: | 2 mM NAD⁺ in buffer, |
| | NAD⁺ (from Sigma) solutions are prepared freshly before each test. |
| | In each case 3 μl of labeled [¹⁴C]-NAD⁺ (2.8 kBq, from Amersham) |
| | are added to 7 μl of cold NAD⁺ solution. |
| Trichloroacetic acid (TCA): | TCA is stored at 4° C. as a 10% strength by weight |
| | solution. |
| PARS: | Human PARS protein is expressed recombinantly in the baculovirus |
| | system (Bac-To-Bac, Baculo virus expression system; Instruction |
| | Manual; Life Technologies) and purified. 500 μl aliquots are stored at |
| | −80° C. |

Methods

The compounds to be tested are dissolved in DMSO (dimethyl sulfoxide) at a concentration of 10 mM. The assay is carried out in deep 96-well plates. Per well, 70 μl of buffer, 10 μl of DNA, 10 μl of histones, 10 μl of NAD⁺/[¹⁴C]-NAD⁺ mix and 0.5-5 μl of PARS (about 10,000 cpm/test) are combined with 1 μl of the compounds (final concentration 0.001-10 μM), to give a total volume of about 110 μl. The mixture is incubated at room temperature for 10 min, and 1 ml of ice-cold TCA solution is then added, and the precipitated labeled proteins are sucked onto a filter paper (printed filter mat A; from Wallac) using a harvester (from Scatron). The filter is dried, sealed together with a scintillation sheet (Multilex A; from Wallac) and measured in a p counter for 1 min per well.

Results of the PARS Inhibition Test

In addition to the substances described in this application, the known PARS inhibitor 1,5-dihydroxyisoquinoline (DHCH) is tested as reference substance. The results of the test are stated as IC₅₀ values for the inhibition of PARS.

The results are shown in Table 1:

TABLE 1
PARS inhibition (in vitro)

| Example | IC₅₀ [nM] |
|---|---|
| DHCH | 300 |
| 1 | 60 |
| 6 | 50 |
| 8 | 85 |
| 40 | 20 |
| 53 | 80 |
| 56 | 75 |
| 62 | 800 |

2) Test Description Cell Protection Assay (In Vitro)

In accordance with a method described by Bowes [Bowes et al., Br. J. Pharmacol., 124, 1760-1766 (1998)], the ability of PARS inhibitors to protect cells against cell death induced by incubation with H₂O₂ is examined in a cell protection assay. Incubation of endothelial cells with H₂O₂ results in the generation of DNA strand breaks which in turn activate PARS, resulting in a drastic energy decrease in the cells and in cell death. Living cells are quantified by a fluorimetric redox indicator (Alamar blue), which is converted in the electron transport system of the mitochondria.

Specifically, 7500 MHEC5-T cells/well (DSM ACC 336; German collection of microorganisms and cell cultures) are sown in 4 replications on a 96-well plate. After 24 hours, the cells are incubated with 3 mM aqueous H₂O₂ solution and differing concentrations of the substances in the presence of 6% by volume of Alamar blue solution in the medium at 37° C. for 5 hours. The reference substance used is 10 μM 1,5-dihydroxyisoquinoline (DHCH) solution. After the incubation, the fluorescence is measured at an excitation wavelength of 530-560 nm and an emission wavelength of 590 nm. The percentage for the cell protection is calculated as the difference between the living cells treated only with H₂O₂ and the cells treated with H₂O₂ and PARS inhibitors. The internal standard used is 10 μM DHCH, which is defined as 100% protection. The results obtained for the other substances are compared to this value.

Results of the Cell Protection Assays:

Examples of the protection of endothelial cells by PARS inhibitors are listed in Table 2 below. The EC₅₀ values indicate the concentration at which 50% of maximum cell protection is reached, maximum protection by 10 μM DHCH being defined as 100%. DHCH has an EC₅₀ value of 2 μM.

TABLE 2
Cell protection (in vitro)

| Example | EC₅₀ [nM] |
|---|---|
| 5 | 150 |
| 61 | 100 |
| 65 | 90 |
| 66 | 650 |
| 76 | 500 |

3) Test Description “Working Heart” Model (In Vivo)

For tests on isolated hearts in the “working heart” mode [Bardenheuer and Schrader, Circulation Res., 51, 263 (1983)], isolated hearts of rats are subjected to a 60-minute “low-flow” phase to generate global ischemia, and the action of the substances with respect to the reestablishment of the pressure in the left ventricle (LVPmax) and the contractile force (dP/dt) during the reperfusion phase is examined. The control substance used is 1,5-dihydroxyisoquinoline.

B. Working Examples:

In the description of the examples, the following abbreviations are used:
DMF=N,N-Dimethylformamide
DMAP=4-Dimethylaminopyridine
EDC=N-(3-Dimethylaminopropyl)-N′-ethylcarbodiimide hydrochloride
HOBt=1-Hydroxy-1H-benzotriazole

### EXAMPLE 1

### 1-[2-(4-Bromophenyl)ethyl]-1,5,7,8-tetrahydro-2H-thiopyrano[4,3-d]pyrimidine-2,4(3H)-dione

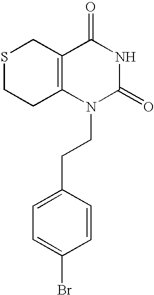

10 g (50.0 mmol) of 4-(bromophenyl)ethylamine and 6.39 g (55.0 mmol) of tetrahydrothiopyran-4-one are initially charged in 250 ml of toluene, a spatula tip of camphor sulfonic acid is added and the mixture is heated under reflux on a water separator for 1.5 hours. The reaction solution is then allowed to cool under argon, and 4.0 ml (50.0 mmol) of chlorocarbonyl isocyanate are added at room temperature. The reaction mixture is heated at 100° C. for one hour and cooled, and the solvent is then removed under reduced pressure. A sodium bicarbonate solution is added to the resulting residue and the mixture is extracted three times with dichloromethane. The organic phase is washed with saturated sodium chloride solution and dried over sodium sulfate. The solvent is then removed under reduced pressure and the resulting residue is crystallized from ethyl acetate. The product is filtered off and washed with diethyl ether. This gives 13.9 g (37.8 mmol, yield: 74% of theory) of a solid.

¹H NMR (300 MHz, DMSO-d₆): δ=2.44-2.55 (m, 2H), 2.73-2.92 (m, 6H), 3.34-3.43 (m, 2H), 3.92 (t, 2H), 7.21 (d, 2H), 7.50 (d, 2H), 11.46 (s, 1H)

MS (ESIpos): m/z=366.7 (M+H)⁺

### EXAMPLE 2

### Methyl 4-[2-(2,4-dioxo-3,4,7,8-tetrahydro-2H-thiopyrano[4,3-d]pyrimidin-1(5H)-ylethyl]benzoate

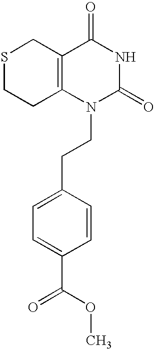

62 mg (0.15 mmol) of 1,3-bis(diphenylphosphino)propane and 31 mg (0.14 mmol) of palladium(II) acetate are initially charged in a flask which had been dried by heating. The flask is flushed with carbon monoxide gas, and a solution of 500 mg (1.36 mmol) of the compound from example 1 in DMF (15 ml) is then transferred into the prepared flask. 1.90 ml (13.6 mmol) of triethylamine and 10 ml (246.8 mmol) of methanol are added, and the reaction mixture is heated at 120° C. After a reaction time of five hours, the solution is allowed to cool and a little water is added. The solution is then extracted three times with ethyl acetate, and the organic phase is washed with water and saturated sodium chloride solution and dried over sodium sulfate. Following filtration and removal of the solvent under reduced pressure, the resulting residue is crystallized from diethyl ether/ethyl acetate. This gives 366 mg (1.1 mmol, yield: 78% of theory) of the title compound.

¹H NMR (300 MHz, DMSO-d₆): δ=2.70-2.90 (m, 4H), 2.95 (t, 2H), 3.35 (s, 2H), 3.83 (s, 3H), 3.97 (t, 2H), 7.40 (d, 2H), 7.91 (d, 2H), 11.43 (s, 1H)

MS (ESIpos): m/z=346.8 (M+H)⁺

### EXAMPLE 3

### 4-[2-(2,4-Dioxo-3,4,7,8-tetrahydro-2H-thiopyrano[4,3-d]pyrimidin-1(5H)-yl)-ethyl]benoic acid

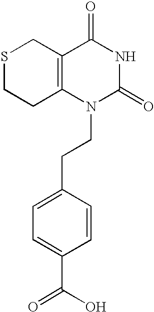

200mg (8.4 mmol) of lithium hydroxide are added to a solution of 580 mg (1.67 mmol) of the compound from example 2 in methanol (27 ml) and water (9 ml), and the reaction mixture is then heated at 50° C. After a reaction time of four hours, the methanol is removed under reduced pressure. The pH of the aqueous phase that remains is adjusted to pH 1 using aqueous hydrochloric acid (6 N) (ice bath cooling). The mixture is then extracted three times with dichloromethane and the organic phase is washed with saturated sodium chloride solution and dried over sodium sulfate. After filtration and removal of the solvent under reduced pressure, the resulting residue is purified by preparative HPLC (column: Kromasil 100 C 18.5 mm; 250×40 mm; mobile phase: acetonitrile/water; flow rate: 50 ml/min; UV detection at 254 nm). This gives 210 mg (0.63 mmol, yield: 38% of theory) of the title compound as a colorless solid.

¹H NMR (300 MHz, DMSO-d₆): δ=2.82 (s, 4H), 2.93 (t, 2H), 3.18 (s, 2H), 3.96 (t, 2H), 7.36 (d, 2H), 7.88 (d, 2H), 11.45 (s, 1H), 12.91 (s, 1H)

MS (ESIpos): m/z 332.7 (M+H)⁺

### EXAMPLE 4

### 1-{2-[4-(1-Piperazinylcarbonyl)phenyl]ethyl}-1,5,7,8-tetrahydro-2H-thiopyrano[4,3-d]pyrimidine-2,4(3H)-dione

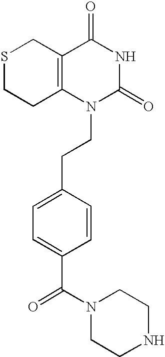

A solution of 50 mg (0.15 mmol) of the compound from example 3, 22 mg (0.17 mmol) of HOBt and 33 mg (0.17 mmol) of EDC in DMF (5 ml) is stirred at room temperature for 30 minutes. 26 mg (0.30 mmol) of piperazine, 46 mg of 4-methylmorpholine (0.45 mmol) and a spatula tip of DMAP are added to this solution, and the mixture is stirred at room temperature overnight. After filtration, the reaction solution is directly separated by preparative HPLC (column: Kromasil 100 C 18.5 mm; 250×40 mm; mobile phase: acetonitrile/water; flow rate: 25 ml/min; UV detection at 254 nm). This gives 11.5 mg (24.4 mmol, yield: 16% of theory) of the title compound.

¹H NMR (300 MHz, DMSO-d₆): δ=2.65-2.70 (m, 6H), 2.70-2.82 (m, 2H), 2.90 (t, 2H), 3.33 (s, 2H), 3.38-3.68 (m, 4H), 3.95 (t, 2H), 7.30 (d, 2H), 8.30 (d, 2H)

MS (ESIpos): m/z=401.3 (M+H)⁺

### EXAMPLE 5

### 4-[2-(2,4-Dioxo-3,4,7,8-tetrahydro-2H-thiopyrano[4,3-d]pyrimidin-1(5H)-ylethyl]benzaldehyde

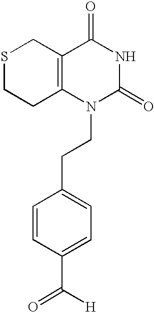

A solution of 1 g (2.7 mmol) of the compound from example 1 in tetrahydrofuran (50 ml) is cooled to −78° C. 3.5 ml (5.58 mmol) of a solution of n-butyllithium (1.6 M) in n-hexane are added dropwise, followed by the addition of 3.1 g (27.2 mmol) of n-formylpiperidine. After the addition, the mixture is warmed to −20° C. and stirred for 18 h. The reaction mixture is then warmed to −10° C., and 10 ml of water are added. The aqueous phase is then extracted three times with dichloromethane and the combined organic phases are washed with saturated sodium chloride solution and dried over sodium sulfate. After removal of the solvent under reduced pressure, the resulting residue is purified by preparative HPLC (column: Kromasil 100 C 18.5 mm; 250×40 mm; mobile phase: acetonitrile/water; flow rate: 50 ml/min; UV detection at 254 nm). This gives 257 mg (0.81 mmol, yield: 30% of theory) of the title compound.

¹H NMR (300 MHz, DMSO-d₆): δ=2.76-2.86 (m, 4H), 2.97 (t, 2H), 3.36 (s, 2H), 3.98 (t, 2H), 7.50 (d, 2H), 7.88 (d, 2H), 9.98 (s, 1H), 11.45 (s, 1H)

MS (ESIpos): m/z=316.8 (M+H)⁺

### EXAMPLE 6

### 1-[2-(4-{[(2-Hydroxyethyl)amino]methyl}phenyl)ethyl]-1,5,7,8-tetrahydro-2H-thiopyrano-[4,3-d]pyrimidine-2,4(3H)-dione

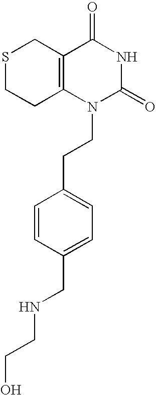

201 mg (0.95 mmol) of sodium triacetoxyborohydride and 0.072 ml (1.26 mmol) of glacial acetic acid are added to a solution of 200 mg (0.63 mmol) of the compound from example 5 and 386 mg (6.3 mmol) of 2-aminoethanol in 1,2-dichloroethane (20 ml). After a reaction time of three hours at room temperature, concentrated aqueous ammonia solution is added and the mixture is then extracted three times with dichloromethane. The combined organic phases are washed with saturated aqueous sodium chloride solution and dried over sodium sulfate. After filtration and removal of the solvent under reduced pressure, the resulting residue is separated by chromatography on silica gel (mobile phase: dichloromethane/methanol/ammonia). This gives 170 mg (0.47 mmol, yield: 74% of theory) of the title compound as a yellow solid.

¹H NMR (300 MHz, DMSO-d₆): δ=2.52 (t, 2H), 2.68-2.89 (m, 6H), 3.33 (s, 2H), 3.45 (t, 2H), 3.68 (s, 2H), 3.90 (t, 2H), 4.42 (s, 1H), 7.16 (d, 2H), 7.24 (d, 2H)

MS (ESIpos): m/z=362 (M+H)⁺

### EXAMPLE 7

### 1-[2-(2,4-Dimethyl-1H-imidazol-1-yl)propyl]-1,5,7,8-tetrahydro-2H-thiopyrano[4,3-d]-pyrimidine-2,4(3H)-dione

### a) 2-[3-(2,4-Dimethyl-1H-imidazol-1-yl)propyl]-1H-isoindole-1,3(2H)-dione

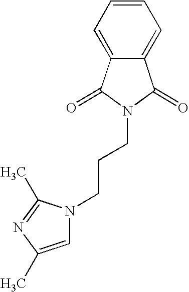

A solution of 2 g (20.8 mmol) of 2,4-dimethylimidazole, 5.9 g (21.8 mmol) of 3-(bromopropyl)phthalimide and 3 g (21.8 mmol) of potassium carbonate in DMF (30 ml) is heated at 110° C. for 2 h. After cooling to room temperature, a little water is added and the mixture is extracted three times with ethyl acetate. The organic phase is then washed with saturated aqueous sodium chloride solution and dried over sodium sulfate. Removal of the solvent under reduced pressure gives a residue which is directly reacted further in the next step.

### b) 3-(2,4-Dimethyl-1H-imidazol-1-yl)propylamine hydrochloride

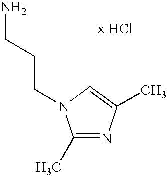

8.8 ml (42.7 mmol) of a 24% strength aqueous hydrazine hydride solution are added to a solution of the 2-[3-(2,4-dimethyl-1H-imidazol-1-yl)propyl]-1H-isoindole-1,3(2H)-dione (crude product) obtained in preparation step 1 in ethanol (50 ml), and the reaction mixture is heated at reflux temperature for 2 h. After cooling of the reaction mixture to room temperature and reduction of the solvent under reduced pressure to about a third of its volume, the resulting precipitate is filtered off and washed with a little ethanol. Concentrated aqueous hydrochloric acid (50 ml) is added to the resulting filtrate, the precipitated solid is filtered off and washed with conc. aqueous hydrochloric acid and the resulting filtrate is removed completely from the solvent under reduced pressure. This gives 1.2 g (6.5 mmol, yield: 31% of theory) of the title compound which is directly, without further purification, reacted in the next step.

¹H NMR (200 MHz, DMSO-d₆): δ=2.05 (t, 2H), 2.21 (s, 2H), 2.58 (s, 3H), 2.62-2.94 (m, 2H), 4.19 (t, 2H), 7.41 (s, 1H)

MS (ESIpos): m/z=154.1 (M+H)⁺

### c) 1-[3-(2,4-Dimethyl-1H-imidazol-1-yl)propyl]-1,5,7,8-tetrahydro-2H-thiopyrano[4,3-d]pyrimidine-2,4(3H)-dione

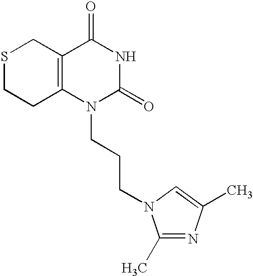

A solution of 1.2 g (6.5 mmol) of 3-(2,4-dimethyl-1H-imidazol-1-yl)propylamine hydrochloride (see preparation step 2) in about 100 ml of saturated aqueous sodium bicarbonate solution is concentrated to dryness under reduced pressure (liberation of the amine), the residue is suspended in 50 ml of toluene, 663 mg (5.7 mmol) of tetrahydrothiopyran-4-one and a spatula tip of camphorsulfonic acid are added and the mixture is heated under reflux on a water separator for 1.5 hours. The reaction mixture is then allowed to cool to room temperature, and 0.5 ml (6.2 mmol) of chlorocarbonyl isocyanate is added. The reaction mixture is again heated at 100° C. for one hour, and, after cooling of the reaction mixture, the solvent is then removed under reduced pressure. The resulting residue is chromatographed on silica gel (mobile phase: dichloromethane/methanol/ammonia). This gives 590 mg (1.8 mmol, yield: 32% of theory) of the title compound as a beige solid.

¹H NMR (400 MHz, DMSO-d₆): δ=1.91 (t, 2H), 2.00 (s, 3H), 2.21 (s, 3H), 2.78 (t, 2H), 2.84 (t, 2H), 3.27-3.49 (m, 2H), 3.73 (t, 2H), 3.84 (t, 2H), 6.77 (s, 1H), 11.41 (s, 1H)

MS (ESIpos): m/z =321 (M+H)⁺

### EXAMPLE 8

### 1-[3-(4-Methyl-1H-imidazol-1-yl)propyl]-1,5,7,8-tetrahydro-2H-thiopyrano[4,3-d]pyrimidine-2,4(3H)-dione

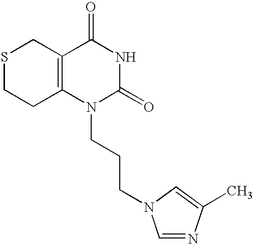

Analogously to the procedure for examples 7 a) and b), 3-(4-methyl-1H-imidazol-1-yl)-1-propaneamine hydrochloride is prepared in two steps from 5 g (60.9 mmol) of 4-methyl-1H-imidazole (3.78 g, 21.5 mmol, yield: 35% of theory). Analogously to the procedure for example 7 c), after liberation of the amine and reaction with tetrahydrothiopyran-4-one (2.2 g, 18.97 mmol) and chlorocarbonyl isocyanate (1.67 ml, 20.69 mmol) a crude product is obtained which is purified by preparative HPLC chromatography (column: Kromasil 100 C 18, 250×20 mm; mobile phase: methanol/0.2% strength aqueous trifluoroacetic acid; flow rate: 25 ml/min; UV detection at 274 nm). This gives 125 mg (0.4 mmol, yield: 2%. of theory) of the title compound as a free base and 304 mg (0.7 mmol, yield: 4% of theory) as the corresponding trifluoroacetate salt.

Analysis of the free base:

¹H NMR (300 MHz, DMSO-d₆): δ=1.98 (t, 2H), 2.08 (s, 3H), 2.75 (t, 2H), 2.85 (t, 2H), 3.23-3.48 (m, 2H), 3.75 (t, 2H), 3.95 (t, 2H), 6.92 (s, 1H), 7.59 (s, 1H), 11.49 (s, 1H)

MS (ESIpos): m/z=307 (M+H)⁺

### EXAMPLE 9

### 1-[2-(4-Nitrophenyl)ethyl]-1,5,7,8-tetrahydro-2H-thiopyrano [4,3-d]pyrimidine-2,4(3H)-dione

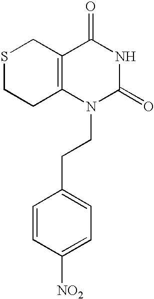

A spatula tip of camphorsulfonic acid is added to a solution of 5.7 g (28.1 mmol) of 4-nitrophenylethylamine and 3.6 g (30.9 mmol) of tetrahydrothiopyran-4-one in 100 ml of toluene, and the mixture is heated under reflux on a water separator for 1.5 hours. The reaction solution is then allowed to cool under argon, and 2.7 ml (33.8 mmol) of chlorocarbonyl isocyanate are added at room temperature. The reaction mixture is heated at 100° C. for one hour and, after cooling to room temperature, the solvent is then removed under reduced pressure. The reaction mixture is triturated with water (about 100 ml) and the precipitated solid is filtered off, washed with a little water/diethyl ether and then dried under reduced pressure. This gives 7.3 g (21.9 mmol, yield: 75% of theory) of the title compound as a colorless solid.

¹H NMR (300 MHz, DMSO-d₆): δ=2.78-2.90 (m, 4H), 3.01 (t, 2H), 3.30-3.42 (m, 2H), 3.99 (t, 2H), 7.55 (d, 2H), 8.18 (d, 2H), 11.43 (s, 1H)

MS (ESIpos): m/z=333.8 (M+H)⁺

### EXAMPLE 10

### 1-[2-(4-Aminophenyl)ethyl]-1,5,7,8-tetrahydro-2H-thiopyrano[4,3-d]pyrimidine-2,4(3H)-dione

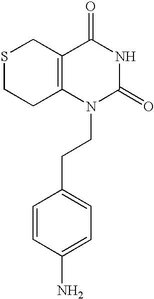

320 mg (0.30 mmol) of palladium (10% on activated carbon) are added to a suspension of 1 g (3 mmol) of the compound from example 9 in methanol (100 ml) and tetrahydrofuran (100 ml). The reaction mixture is hydrogenated in a hydrostatic hydrogen atmosphere overnight. The mixture is then filtered through Celite, and the filter cake is washed with methanol. The solvent is removed under reduced pressure and the resulting residue is purified by preparative HPLC (column: Kromasil 100 C 18.5 mm; 250×40 mm; mobile phase: acetonitrile/water; flow rate: 50 ml/min; UV detection at 254 nm). This gives 417 mg (1.37 mmol, yield: 45% of theory) of the title compound as a yellow solid.

¹H NMR (300 MHz, DMSO-d₆): δ=2.65 (t, 2H), 2.72-2.82 (m, 4H), 3.29-3.40 (m, 1H), 3.82 (t, 2H), 4.92 (s, 2H), 6.49 (d, 2H), 6.86 (d, 2H), 11.42 (s, 1H)

MS (ESIpos): m/z=304 (M+H)⁺

### EXAMPLE 11

### N-{4-[2-(2,4-Dioxo-3,4,7,8-tetrahydro-2H-thiopyrano[4,3-d]pyrimidine-1(5H)-yl)ethyl]phenyl-n-tert-butyloxycarbonyl glycinamide

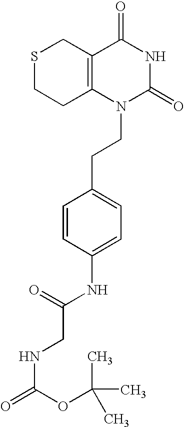

A solution of 173 mg (0.99 mmol) of N-(tert-butoxycarbonyl)glycine, 98 mg (0.73 mmol) of HOBt and 145 mg (0.76 mmol) of EDC in DMF (5 ml) is stirred at room temperature for 30 minutes. 200 mg (0.66 mmol) of the compound from example 10, 0.22 ml (2.0 mmol) of 4-methylmorpholine and a spatula tip of DMAP are added to this solution, and the mixture is stirred at room temperature overnight. After filtration, the reaction solution is directly separated by preparative HPLC (column: Kromasil 100 C 18.5 mm; 250×40 mm; mobile phase: acetonitrile/water; flow rate: 50 ml/min; UV detection at 254 nm). This gives 243 mg (0.53 mmol, yield: 80% of theory) of the title compound as a colorless solid.

¹H NMR (300 MHz, DMSO-d₆): δ=1.40 (s, 9H), 2.68-2.85 (m, 6H), 3.43 (m, 2H), 3.63-3.75 (m, 2H), 3.90 (t, 2H), 7.00 (t, 1H), 7.18 (d, 2H), 7.51 (d, 2H), 9.90 (s, 1H)

MS (ESIpos): m/z=461 (M+H)⁺

### EXAMPLE 12

### N-{4-[2-(2,4-Dioxo-3,4,7,8-tetrahydro-2H-thiopyrano[4,3-d]pyrimidin-1(5H)-yl)ethyl]phenylglycinamide

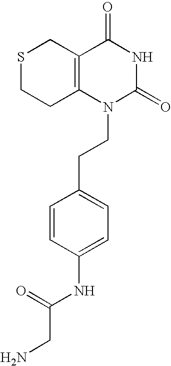

Trifluoroacetic acid (2 ml) is added to a solution of 200 mg (0.43 mmol) of the compound from example 11 in dichloromethane (4 ml), the mixture is stirred at room temperature for one hour, a little water is added and the mixture is extracted three times with dichloromethane. The combined organic phases are then washed with saturated aqueous sodium chloride solution and dried over sodium sulfate. After filtration and removal of the solvent under reduced pressure, the resulting residue is purified by preparative HPLC (column: Kromasil 100 C 18.5 mm; 250×40 mm; mobile phase: acetonitrile/water; flow rate: 50 ml/min; UV detection at 254 nm). This gives 46 mg (0.13 mmol, yield: 29% of theory) of the title compound as a colorless solid.

¹H NMR (300 MHz, DMSO-d₆): δ=2.69-2.89 (m, 6H), 3.14-3.55 (m, 4H), 3.9 (t, 2H), 7.16 (d, 2H), 7.57 (d, 2H)

MS (ESIpos): m/z=361 (M+H)⁺

### EXAMPLE 13

### 1-[2-(4-Nitrophenyl)ethyl]-5,6,7,8-tetrahydro-2,4(1H,3H)-quinazolinedione

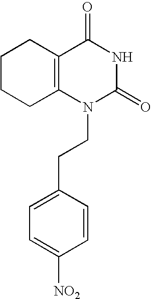

A spatula tip of camphorsulfonic acid is added to a mixture of 7.35 g (44.2 mmol) of 2-(4-nitrophenyl)ethylamine and 4.3 g (44.2 mmol) of cyclohexanone in 300 ml of toluene and the mixture is heated under reflux on a water separator for 3 hours. The reaction mixture is then allowed to cool, and 3.6 ml (44.2 mmol) of chlorocarbonyl isocyanate are added at room temperature. The reaction mixture is heated at 130° C. for one hour and, after cooling to room temperature, the solvent is removed under reduced pressure. The resulting residue is crystallized from ethyl acetate, and the solid is filtered off with suction, washed with diethyl ether and dried under high vacuum. This gives 10.2 g (32.3 mmol, yield: 73% of theory) of the title compound as a yellowish solid.

¹H NMR (300 MHz, DMSO-d₆): δ=1.44-1.74 (m, 4H), 2.20 (t, 2H), 2.42-2.59 (m, 2H), 3.00 (t, 2H), 3.96 (t, 2H), 7.52 (d, 2H), 8.19 (d, 2H), 11.25 (s, 1H)

MS (ESIpos): m/z=316 (M+H)⁺

### EXAMPLE 14

### 1-[2-(4-Aminophenyl)ethyl]-5,6,7,8-tetrahydro-2,4(1H,3H)-quinazolinedione

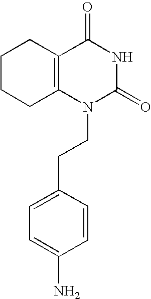

180 mg of palladium (10% on activated carbon) are added to a suspension of 1.8 g (5.7 mmol) of the compound from example 13 in methanol (45 ml) and tetrahydrofuran (90 ml), and the mixture is hydrogenated under a hydrostatic hydrogen atmosphere overnight. After filtration through Celite and washing of the filter cake with methanol, the solvent is removed under reduced pressure. The resulting residue is recrystallized from ethyl acetate and the solid is filtered off, washed with diethyl ether and dried under high vacuum. This gives 1.0 g (3.5 mmol, yield: 61% of theory) of the title compound as a colorless solid.

¹H NMR (300 MHz, DMSO-d₆): δ=1.40-1.70 (m, 4H), 2.18 (t, 2H), 2.40 (t, 2H), 2.64 (t, 2H), 3.79 (t, 2H), 4.92 (s, 2H), 6.49 (d, 2H), 6.82 (d, 2H), 11.20 (s, 1H)

MS (ESIpos): m/z=286 (M+H)⁺

### EXAMPLE 15

### N-{4-[2-(2,4-Dioxo-3,4,5,6,7,8-hexahydro-1(2H)-quinazolinyl)ethyl]phenyl}-2-thiophenesulfonamide

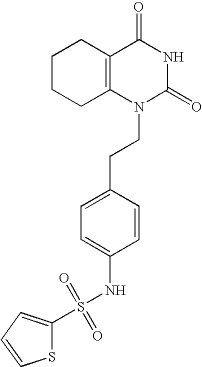

32 mg (0.18 mmol) of 2-thiophenesulfonyl chloride are added to a solution of 50 mg (0.18 mmol) of the compound from example 14 in pyridine (2.5 ml), and the mixture is stirred at room temperature overnight. A little water is then added to the reaction mixture, the pH is adjusted to neutral using aqueous hydrochloric acid (2 N) and the mixture is extracted three times with dichloromethane. The combined organic phases are washed with water and saturated sodium chloride solution and dried over sodium sulfate. Removal of the solvent under reduced pressure gives 32.5 mg (0.8 mmol, yield: 40% of theory) of the title compound as a colorless solid.

¹H NMR (300 MHz, DMSO-d₆): δ=1.38-1.59 (m, 4H), 2.15 (t, 2H), 2.25-2.38 (m, 2H), 2.76 (t, 2H), 3.82 (t, 2H), 7.00-7.14 (m, 5H), 7.51 (d, 1H), 7.89 (s, 1H), 10.37 (s, 1H), 11.21 (s, 1H)

MS (ESIpos): m/z=432.2 (M+H)⁺

Working examples 16 to 82 listed in the table below were prepared analogously to examples 1 to 15 described above:

| Example | calc. | | Rₜ (min.)/ | mass found, |
|---|---|---|---|---|
| No. | mass | Structure | HPLC-method | m/z (M + H)⁺ |
| 16 | 314.39 | 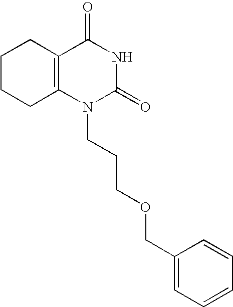 | 3.63 (B) | 315.2 |
| 17 | 257.29 | 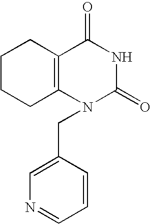 | 2.54 (E) | 258 |
| 18 | 330.39 | 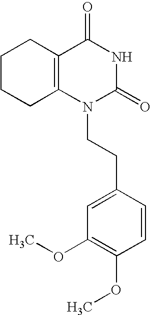 | 3.70 (E) | 331 |
| 19 | 286.33 | 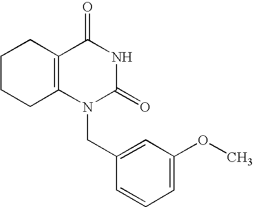 | 3.82 (E) | 287 |
| 20 | 284.36 | 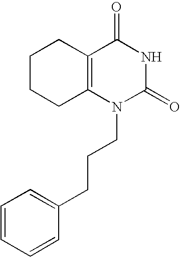 | 3.70 (B) | 285.4 |
| 21 | 274.33 | 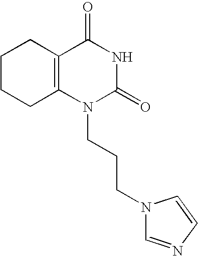 | 2.97 (A) | 275 |
| 22 | 275.31 | 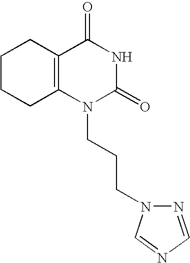 | 3.03 (A) | 276 |
| 23 | 288.35 | 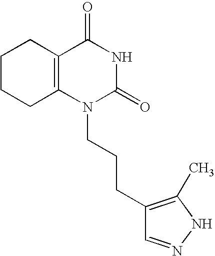 | 2.29 (B) | 289.1 |
| 24 | 298.38 | 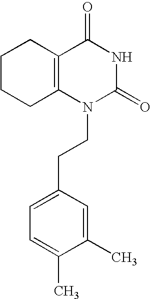 | 3.98 (B) | 299.1 |
| 25 | 300.36 | 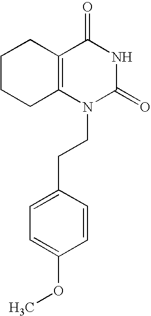 | 4.08 (A) | 301 |
| 26 | 328.41 | 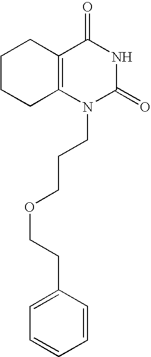 | 3.81 (B) | 329.2 |
| 27 | 320.39 | 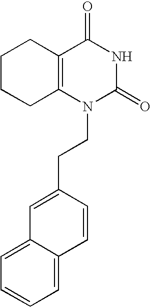 | 3.93 (B) | 321.1 |
| 28 | 315.33 | 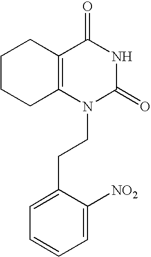 | 3.40 (B) | 316.1 |
| 29 | 298.38 | 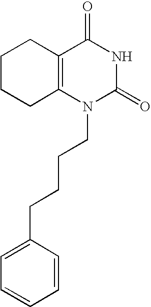 | 3.94 (B) | 299.1 |
| 30 | 271.32 | 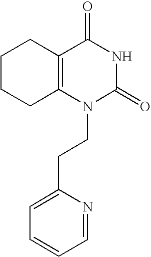 | 0.46 (B) | 372.1 |
| 31 | 349.23 | 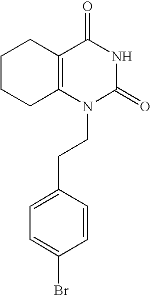 | 4.20 (C) | 349 |
| 32 | 328.37 | 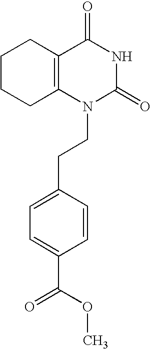 | 3.40 (B) | 329.1 |
| 33 | 271.32 | 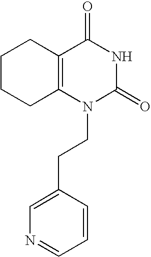 | 0.32 (B) | 272.2 |
| 34 | 347.37 | 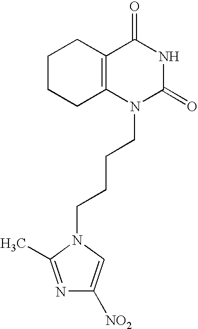 | 3.45 (A) | 348.2 |
| 35 | 329.35 | 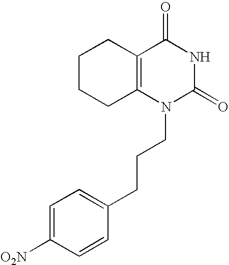 | 3.59 (B) | 330.1 |
| 36 | 377.46 | 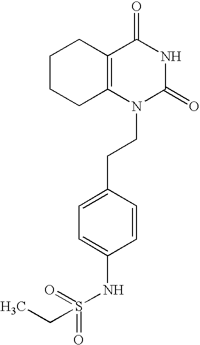 | 3.08 (B) | 378.2 |
| 37 | 363.44 | 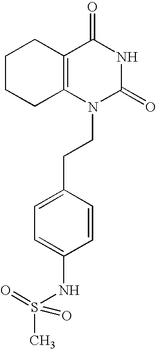 | 2.88 (B) | 364.2 |
| 38 | 333.35 | 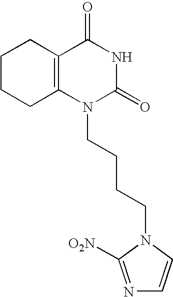 | 3.14 (A) | 334.1 |
| 39 | 317.30 | 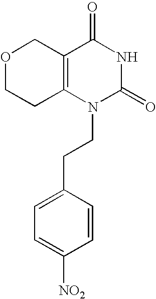 | 3.64 (A) | 318 |
| 40 | 333.37 | 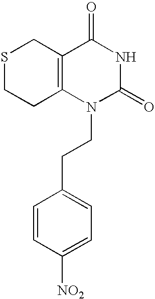 | 3.91 (A) | 334 |
| 41 | 271.32 | 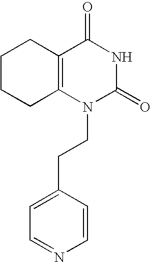 | 2.24 (B) | 270 (M − H)⁺ |
| 42 | 419.54 | 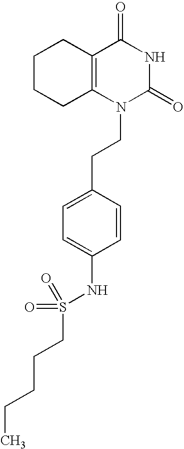 | 4.03 (C) | 420 |
| 43 | 288.37 | 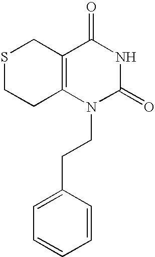 | 3.72 (A) | 289.1 |
| 44 | 349.41 | 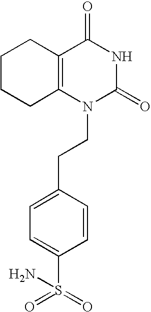 | 3.33 (A) | 350.4 |
| 45 | 367.45 | 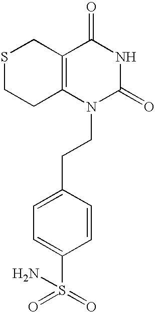 | 3.40 (A) | 368 |
| 46 | 334.44 | 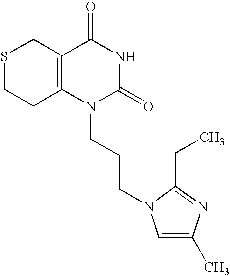 | 0.53 (B) | 335.3 |
| 47 | 365.41 | 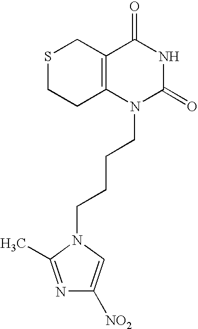 | 3.38 (A) | 383.1 (M + NH₄)⁺ |
| 48 | 351.39 | 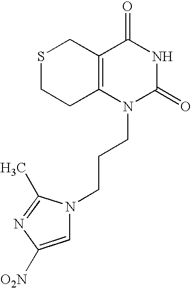 | 3.26 (A) | 352 |
| 49 | 306.39 | 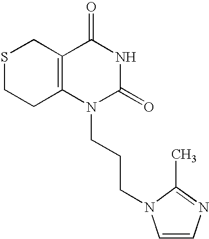 | 0.44 (C) | 307 |
| 50 | 367.27 | 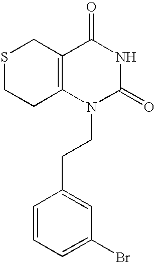 | 4.23 (A) | 367 |
| 51 | 298.34 | 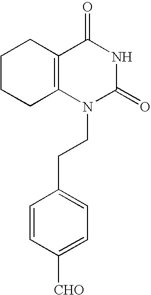 | 3.74 (A) | 299 |
| 52 | 293.35 | 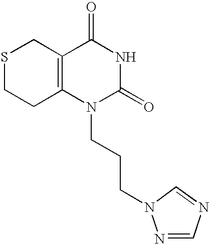 | 2.88 (A) | 294 |
| 53 | 306.39 | 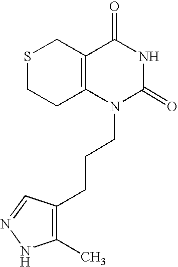 | 3.14 (A) | 307 |
| 54 | 327.43 | 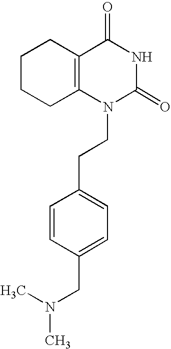 | 3.35 (A) | 328 |
| 55 | 318.40 | 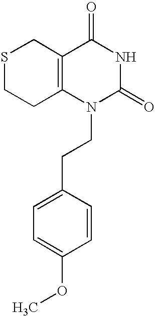 | 3.92 (A) | 319 |
| 56 | 275.33 | 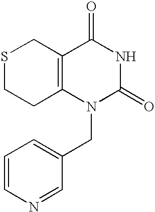 | 2.59 (A) | 276 |
| 57 | 313.40 | 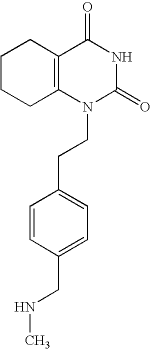 | 3.31 (A) | 314 |
| 58 | 371.50 | 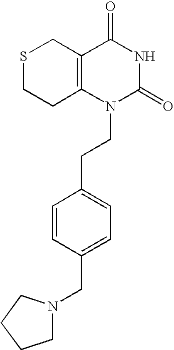 | 2.77 (A) | 372 |
| 59 | 345.46 | 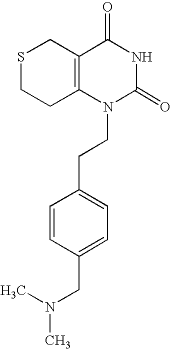 | 0.83 (B) | 346.2 |
| 60 | 331.44 | 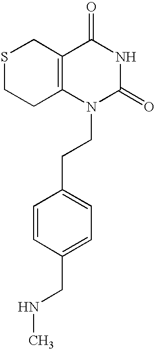 | 0.81 (B) | 332.3 |
| 61 | 385.53 | 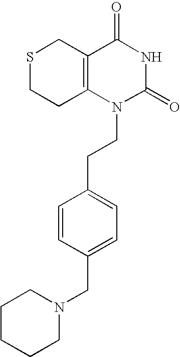 | 3.44 (A) | 386 |
| 62 | 442.58 | 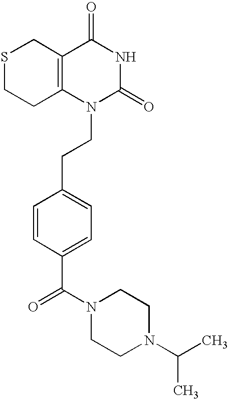 | 3.33 (A) | 443 |
| 63 | 387.50 | 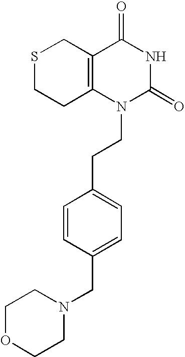 | 3.30 (A) | 388 |
| 64 | 303.38 | 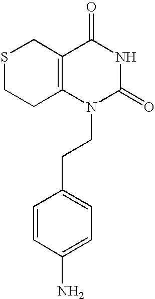 | 3.05 (A) | 304 |
| 65 | 337.36 | 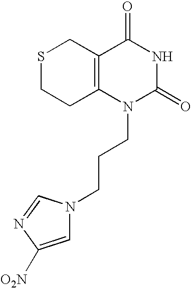 | 3.19 (A) | 333 |
| 66 | 289.36 | 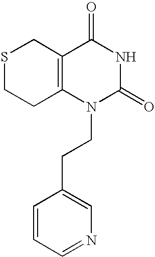 | 2.89 (A) | 290 |
| 67 | 289.36 | 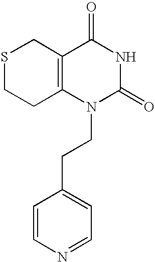 | 2.87 (A) | 290 |
| 68 | 430.57 | 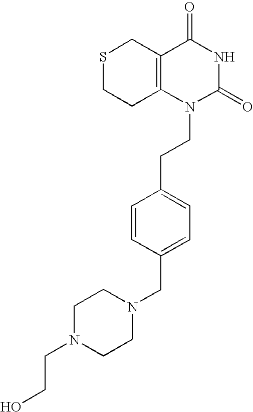 | 3.20 (A) | 431 |
| 69 | 443.57 | 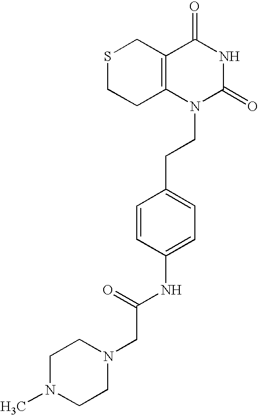 | 3.30 (A) | 444 |
| 70 | 388.49 | 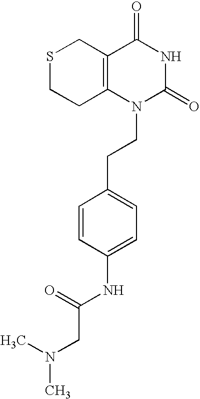 | 3.30 (A) | 389.1 |
| 71 | 529.66 | 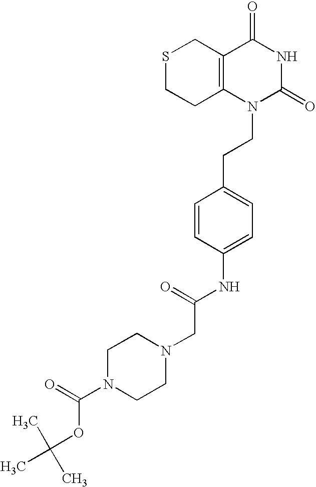 | 3.80 (A) | 530.4 |
| 72 | 368.46 | 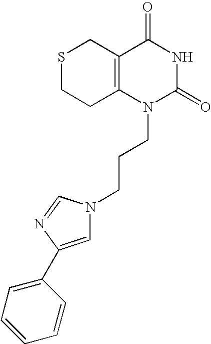 | 3.57 (A) | 369 |
| 73 | 444.55 | 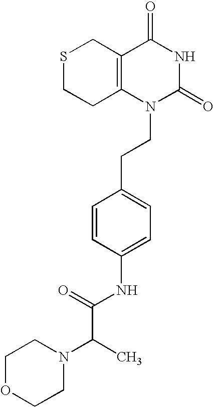 | 3.38 (A) | 445 |
| 74 | 400.54 | 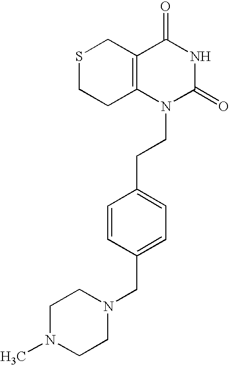 | 3.20 (A) | 401 |
| 75 | 429.54 | 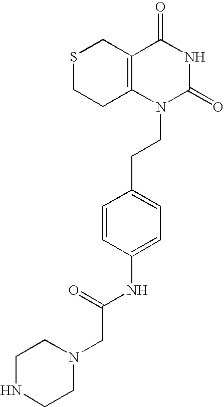 | 3.27 (A) | 430 |
| 76 | 292.36 | 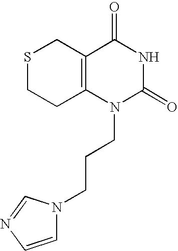 | 2.86 (A) | 293 |
| 77 | 320.42 | 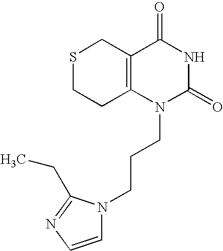 | 3.06 (A) | 321 |
| 78 | 348.47 | 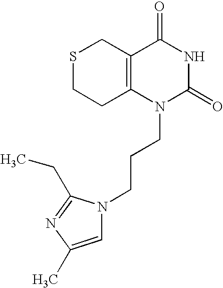 | 3.28 (A) | 349 |
| 79 | 334.44 | 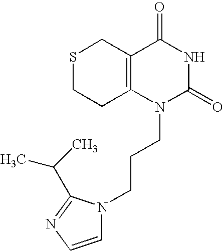 | 3.27 (A) | 335 |
| 80 | 357.48 | 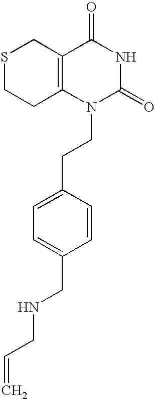 | 3.41 (A) | 358.4 |
| 81 | 382.49 | 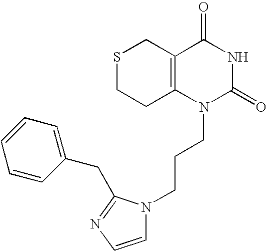 | 3.50 (A) | 383.3 |
| 82 | 340.83 | 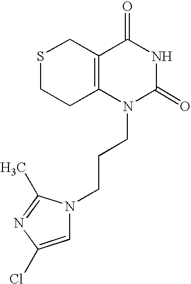 | 3.05 (A) | 341 |

HPLC Methods:
(A): Mobile phase A: 0.5% HClO₄ in water; mobile phase B: acetonitrile; gradient: 0.5 min 98% A, 2% B; 4.5 min 10% A, 90% B; 6.7 min 98% A, 2% B; flow rate: 0.75 ml/min; column temperature: 30° C.; UV detection at 210 mn; column: Kromasil C18 (60×2 mm).
(B): Mobile phase A: 0.1% formic acid in water; mobile phase B: 0.1% formic acid in acetonitrile; gradient: 0 min 90% A, 10% B; 4 min 10% A, 90% B; 6.1 min 90% A, 10% B; flow rate: 0.5 ml/min; column temperature: 40° C.; UV detection at 210 nm; column: Symmetry C18 (150×2.1 mm).
(C): Mobile phase A: 0.06% HCl in water; mobile phase B: acetonitrile; gradient: 1 min 90% A, 10% B; 4 min 10% A, 90% B; flow rate: 0.6 ml/min; column temperature: 50° C.; UV detection at 210 nm; column: Symmetry C18 (150×2.1 mm).
(D): As for method (A), but using the following gradient: 0.5 min 98% A, 2% B; 4.5 min 10% A, 90% B; 9.2 min 98% A, 2% B.
(E): Mobile phase A: 0.01% HCl in water; mobile phase B: acetonitrile; gradient: 0 min 98% A, 2% B; 2.5 min 5% A, 9% B; flow rate: 0.9-1.2 ml/min; column temperature: 70° C.; UV detection at 210 nm; column: Symmetry C18 (150×2.1 mm).
   C. Working Examples of Pharmaceutical Compositions

The compounds according to the invention can be converted into pharmaceutical preparations as follows:

Tablet:

Composition:

100 mg of the compound from example 1, 50 mg of lactose (monohydrate), 50 mg of corn starch (native), 10 mg of polyvinylpyrrolidone (PVP 25) (from BASF, Ludwigshafen, Germany) and 2 mg of magnesium stearate. Tablet weight 212 mg. Diameter 8 mm, radius of the curvature 12 mm.

Preparation:

The mixture of active compound, lactose and starch is granulated using a 5% by weight strength solution of the PVP in water. After drying, the granules are mixed with the magnesium stearate for 5 min. This mixture is tabletted in a customary tablet press (dimensions of the tablet see above). As a guideline for tabletting, a compaction force of 15 kN is used.

Orally Administrable Suspension:

Composition:

1000 mg of the compound of example 1, 1000 mg of ethanol (96%), 400 mg of Rhodigel (xanthan gum from FMC, Pennsylvania, USA), and 99 g of water. A single dose of 100 mg of the compound according to the invention corresponds to 10 ml of oral suspension.

Preparation:

The Rhodigel is suspended in ethanol and the active compound is added to the suspension. The water is added with stirring. The mixture is stirred for about 6 h, until the swelling of the Rhodigel is complete.

1. A compound of the formula (I) 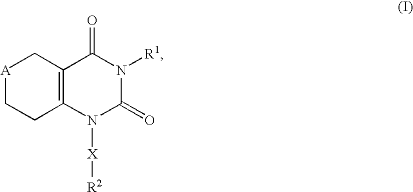
in which
A represents —CH₂—, —O— or —S—,
R¹ represents hydrogen or alkoxycarbonyl,
R² represents aryl or heteroaryl which for their part may be substituted up to three times, independently of one another, by substituents selected from the group consisting of nitro, halogen, cyano, aryl, hetaryl, benzyl, alkyl, cycloalkyl, alkoxy, formyl, alkoxycarbonyl, trifluoromethyl, di- and trifluoromethoxy, hydroxyl, amino, alkylamino, aminosulfonyl, alkylsulfonylamino, arylsulfonylamino, hetarylsulfonylamino, —Y—OR³ and —Y—NR³R⁴,
in which
Y represents CH₂, C(═O) or *—NH—C(═O)—CHR⁵—,
in which * represents the point of attachment to the aromatic or heteroaromatic radical,
R³ and R⁴ independently of one another represent hydrogen, optionally hydroxyl- or amino-substituted alkyl, alkenyl or alkoxycarbonyl,
or
R³ and R⁴ together with the nitrogen atom to which they are attached form a 5- to 7-membered heterocycle which may contain a further heteroatom N, O or S in the ring and which is optionally substituted by amino, hydroxyl, alkoxycarbonyl or alkyl which for its part may be substituted by hydroxyl or amino,
R⁵ represents hydrogen or alkyl which for its part may be substituted by phenyl, 4-hydroxyphenyl, amino, hydroxyl, carboxyl, guanidino, imidazolyl, indolyl, mercapto or methylthio,
or
R³ and R⁵ together represent propane-1,3-diyl or butane-1,4-diyl, and
X represents alkanediyl in which one methylene group may be replaced by an oxygen atom
or a salt, a solvate or a solvate of a salt thereof.

2. A compound as claimed in claim 1,
in which
A represents —CH₂— or —S—,
R¹ represents hydrogen,
R² represents phenyl, pyridyl, pyrazolyl or imidazolyl which for their part may be substituted up to three times, independently of one another, by substituents selected from the group consisting of nitro, halogen, phenyl, benzyl, (C₁-C₄)-alkyl, (C₁-C₄)-alkoxy, formyl, (C₁-C₄)-alkoxycarbonyl, amino, hydroxyl, aminosulfonyl and —Y—NR³R⁴,
in which
Y represents CH₂, *—NH—C(═O)—CH₂— or *—NH—C(═O)—CH(CH₃)—,
in which * represents the point of attachment to the aromatic or heteroaromatic radical,
R³ and R⁴ independently of one another represent hydrogen, optionally hydroxyl- or amino-substituted (C₁-C₄)-alkyl, (C₂-C₄)-alkenyl or (C₁-C₄)-alkoxycarbonyl
or
R³ and R⁴ together with the nitrogen atom to which they are attached form a 5- to 7-membered heterocycle which may contain a further heteroatom N or O in the ring and which is optionally substituted by amino, hydroxyl, (C₁-C₄)-alkoxycarbonyl or (C₁-C₄)-alkyl which for its part may be substituted by hydroxyl or amino,
and
X represents (C₁-C₄)-alkanediyl
or a salt, a solvate or a solvate of a salt thereof.

3. A compound as claimed in claim 1,
in which
A represents —S—,
R¹ represents hydrogen,
R² represents phenyl or imidazolyl which for their part may be substituted up to three times, independently of one another, by substituents selected from the group consisting of nitro, fluorine, chlorine, bromine, methyl, ethyl, isopropyl, methoxycarbonyl and —Y—NR³R⁴,
in which
Y represents CH₂ or *—NH—C(═O)—CH₂—,
in which * represents the point of attachment to phenyl or imidazolyl,
R³ and R⁴ independently of one another represent hydrogen, methyl, ethyl, isopropyl, which are optionally substituted by hydroxyl or amino, or represent allyl or methoxycarbonyl, or
R³ and R⁴ together with the nitrogen atom to which they are attached represent pyrrolidin-1-yl, piperidin-1-yl, piperazin-1-yl, 4-methylpiperazin-1-yl, 4-(2-hydroxyethyl)piperazin-1-yl or morpholin-4-yl
and
X represents ethane-1,2-diyl, propane-1,3-diyl or butane-1,4-diyl
or a salt, a solvate or a solvate of a salt thereof.

4. (canceled)

5. A process for preparing compounds of the formula (I) as defined in claim 1, characterized in that
compounds of the formula (II) 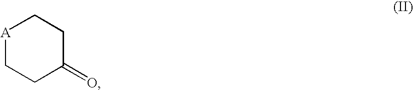
in which
A is as defined in claim 1,
are reacted with compounds of the formula (III)
H₂N—X—R (III),
in which
X and R² are as defined in claim 1,
to give compounds of the formula (IV) 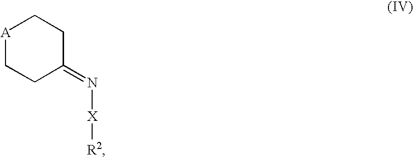
in which
A, X and R are as defined in claim 1,
then reacted with chlorocarbonyl isocyanate to give compounds of the formula (Ia) 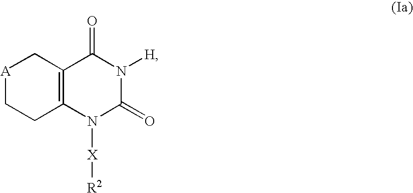
in which
A, X and R² are as defined in claim 1 and R¹ represents hydrogen,
and compounds of the formula (Ia) are, if appropriate, reacted with compounds of the formula (V)
R¹-Z (V),
in which
R¹ is as defined in claim 1, but is not hydrogen, and Z represents a leaving group,
to give compounds of the formula (D) in which R¹ is not hydrogen.

6. A composition, comprising at least one compound of the formula (I) as defined in claim 1 and at least one further active compound.

7. A composition, comprising at least one compound of the formula (I) as defined in claim 1 and one or more pharmaceutically acceptable auxiliaries.

8. A method of treating ischemia and reperfusion damage, comprising administering to a patient in need thereof an effective amount of a compound of formula (I) as defined in claim 1.
